(19) United States
(12) Patent Application Publication
(10) Pub. No.: US 2020/0037989 A1
(43) Pub. Date: Feb. 6, 2020

(54) ULTRASOUND ENDOSCOPE

(71) Applicant: OLYMPUS CORPORATION, Tokyo (JP)

(72) Inventors: Yuko TANIGUCHI, Tokyo (JP); Katsuhiro WAKABAYASHI, Tokyo (JP); Satoshi YOSHIDA, Kawagoe-shi (JP); Sunao SATO, Yamato-shi (JP); Takuya IMAHASHI, Kawasaki-shi (JP)

(73) Assignee: OLYMPUS CORPORATION, Tokyo (JP)

(21) Appl. No.: 16/591,709

(22) Filed: Oct. 3, 2019

Related U.S. Application Data
(63) Continuation of application No. PCT/JP2018/014328, filed on Apr. 3, 2018.

(30) Foreign Application Priority Data
Apr. 3, 2017 (JP) 2017-073879

(51) Int. Cl. A61B 8/00; B06B 1/06; B06B 1/02; A61B 8/12

(52) U.S. Cl. CPC A61B 8/445; B06B 1/0625; B06B 2201/76; A61B 8/12; A61B 8/4494; B06B 1/0215

(57) ABSTRACT

An ultrasound endoscope includes: an insertion part including a distal-end rigid part, a curve part joined to a proximal end side of the distal-end rigid part, and a flexible tube part joined to a proximal end side of the curve part; an ultrasound transducer in which a plurality of piezoelectric elements capable of transmitting and receiving ultrasound are arranged annularly along a circumferential direction of the distal-end rigid part; an imaging sensor provided in the distal-end rigid part; an ultrasound cable including a plurality of coaxial cores that are electrically connected to the piezoelectric elements, respectively, and a metallic integration shield that covers the coaxial cores, the ultrasound cable being fixed to a proximal end side of the distal-end rigid part and on a side of an outer circumference; and a flexible substrate configured to electrically connect the piezoelectric elements and the coaxial cores to each other.

### CROSS-REFERENCE TO RELATED APPLICATION

This application is a continuation of PCT International Application No. PCT/JP2018/014328 filed on Apr. 3, 2018, which designates the United States, incorporated herein by reference, and which claims the benefit of priority from Japanese Patent Application No. 2017-073879, filed on Apr. 3, 2017, incorporated herein by reference.

### BACKGROUND

### 1. Technical Field

The present disclosure relates to an ultrasound endoscope including a radial ultrasound transducer that emits ultrasound to an object to be observed, receives ultrasound echoes that are reflected by the object to be observed, converts the ultrasound echoes into an echo signal, and outputs the echo signal; and an optical system for internal observation of a subject.

### 2. Related Art

Ultrasound may be used in order to observe features of living tissue or a material that is an object to be observed. Specifically, an ultrasound observation apparatus performs given signal processing on the ultrasound echoes that are received from the ultrasound transducer that transmits and receives ultrasound, thereby enabling acquisition of information on the features of the object to be observed.

The ultrasound transducer includes a plurality of piezoelectric elements that convert an electric pulse signal into ultrasound pulses (acoustic pulses), apply the ultrasound pulses to the object to be observed, convert ultrasound echoes reflected by the object to be observed into an electric echo signal, and output the echo signal. For example, the piezoelectric elements are arranged along a given direction and, by switching among the elements relating to transmission and reception electrically and delaying transmission and reception of each element, ultrasound echoes are acquired from the object to be observed.

It has been known that there are multiple different types of ultrasound transducers, such as convex, linear, and radial transducers, whose corresponding areas to be scanned by ultrasound are different from one another. A radial ultrasound transducer includes a plurality of piezoelectric elements that are arranged around a given axis and emits ultrasound beams in a radial direction orthogonal to the axis. For example, Japanese Laid-open Patent Publication No. 2002-153469 discloses an ultrasound endoscope that includes an insertion unit into which a forward viewing optical system for internal observation of a subject, which is an insertion unit including a radial ultrasound transducer at its distal end, and a channel that has a distal end from which a treatment tool is caused to protrude and that sucks a fluid, such as the liquid or gas in the subject are inserted. The ultrasound endoscope disclosed by Japanese Laid-open Patent Publication No. 2002-153469 includes a flexible substrate in which an interconnection pattern is formed is provided around the forward viewing optical system and the channel. The flexible substrate extends from a proximal end side of the ultrasound transducer to a curve part and is connected to an ultrasound cable at the distal end of a flexible tube that is continuous to the proximal end side of the curve part.

### SUMMARY

In some embodiments, an ultrasound endoscope includes: an insertion part including a distal-end rigid part has rigidity, a curve part that is joined to a proximal end side of the distal-end rigid part and that can be curved in at least one direction, and a flexible tube part that is joined to a proximal end side of the curve part and that has flexibility; an ultrasound transducer in which a plurality of piezoelectric elements capable of transmitting and receiving ultrasound are arranged annularly along a circumferential direction of the distal-end rigid part, the ultrasound transducer being configured to apply the ultrasound in a direction orthogonal to a longitudinal direction of the insertion part; an imaging sensor that is provided in the distal-end rigid part, the imaging sensor being configured to capture an forward-viewing image in the longitudinal direction of the insertion part; an ultrasound cable including a plurality of coaxial cores that are electrically connected to the piezoelectric elements, respectively, and a metallic integration shield that covers the coaxial cores, the ultrasound cable being fixed to a proximal end side of the distal-end rigid part and on a side of an outer circumference; and a flexible substrate configured to electrically connect the piezoelectric elements and the coaxial cores to each other. The flexible substrate includes a first connection part that curves annularly and that is electrically connected to the piezoelectric elements, a second connection part that forms an annular shape that curves to a same side as a side to which the first connection part curves and that is electrically connected to the first connection part and the coaxial cores, and a joint configured to join the first connection part and the second connection part, and the joint has a length of extension along a circumferential direction, which is a length smaller than lengths of extension of the first connection part and the second connection part in a circumferential direction.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an ultrasound endoscope system according to a first embodiment of the present disclosure;

FIG. 2 is a side view schematically illustrating a configuration of a distal end of an insertion part of an ultrasound endoscope according to the first embodiment of the disclosure;

FIG. 3 is a perspective view schematically illustrating the configuration of the distal end of the insertion part of the ultrasound endoscope according to the first embodiment of the disclosure;

FIG. 4 is a cross-sectional view taken along the A-A line represented in FIG. 1;

FIG. 5 is a cross-sectional view taken along the B-B line represented in FIG. 2;

FIG. 6 is a schematic view illustrating a configuration of a flexible substrate of the ultrasound endoscope according to the first embodiment of the disclosure;

FIG. 7 is a developed view of the flexible substrate illustrated in FIG. 6;

FIG. 8 is a cross-sectional view schematically illustrating a configuration of a distal end of an insertion part of an ultrasound endoscope according to a second embodiment of the disclosure;

FIG. 9 is a schematic view illustrating a configuration of a flexible substrate of the ultrasound endoscope according to the second embodiment of the disclosure;

FIG. 10 is a developed view of the flexible substrate illustrated in FIG. 9;

FIG. 11 is a schematic view illustrating a configuration of a flexible substrate of an ultrasound endoscope according to Modification 1 of the second embodiment of the disclosure;

FIG. 12 is a schematic view illustrating a configuration of a flexible substrate of an ultrasound endoscope according to Modification 2 of the second embodiment of the disclosure;

FIG. 13 is a developed view of the flexible substrate illustrated in FIG. 12;

FIG. 14 is a schematic view illustrating a configuration of a flexible substrate of an ultrasound endoscope according to a third embodiment of the disclosure;

FIG. 15 is a plane view in a direction of the arrow C in FIG. 14;

FIG. 16 is a schematic view illustrating a configuration of a flexible substrate of an ultrasound endoscope according to a fourth embodiment of the disclosure;

FIG. 17 is a cross-sectional view schematically illustrating a configuration of a distal end of an insertion part of an ultrasound endoscope according to a fifth embodiment of the disclosure;

FIG. 18 is a schematic view illustrating a mode of connection between a flexible substrate and a cable of the ultrasound endoscope according to the fifth embodiment of the disclosure; and

FIG. 19 is a schematic view illustrating another exemplary configuration of the flexible substrate of the ultrasound endoscope according to an embodiment of the disclosure.

### DETAILED DESCRIPTION

Modes for carrying out the present disclosure (“embodiments” below) will be described below with reference to the accompanying drawings. Note that the embodiments described below do not limit the disclosure. Like parts are denoted with like reference numbers in the drawings.

### First Embodiment

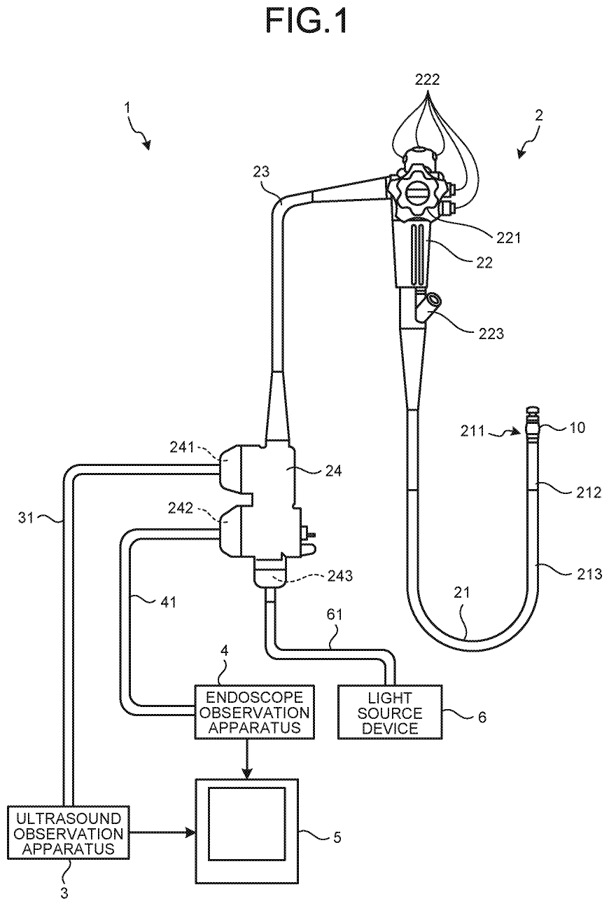

FIG. 1 is a diagram schematically illustrating an ultrasound endoscope system according to a first embodiment of the disclosure. An endoscope system 1 is a system that gives an internal ultrasound diagnosis on a subject, such as a human, using an ultrasound endoscope. As illustrated in FIG. 1, the ultrasound endoscope system 1 includes an ultrasound endoscope 2, an ultrasound observation apparatus 3, an endoscope observation apparatus 4, a display device 5, and a light source device 6.

The ultrasound endoscope 2 is obtained by combining an ultrasound probe with an endoscope observation unit including an observation optical system formed of lenses, etc., and an imaging device. The ultrasound endoscope 2 has an endoscope observation function and an ultrasound observation function. The ultrasound endoscope 2 includes an ultrasound transducer in its distal end. The ultrasound transducer converts an electric pulse signal that is transmitted from the ultrasound observation apparatus 3 into ultrasound pulses (acoustic pulses), applies the ultrasound pulses onto the subject, converts ultrasound echoes that are reflected by the subject into an electric echo signal expressing the ultrasound echoes by a voltage change, and outputs the echo signal. The configuration of the ultrasound transducer will be described below.

The ultrasound endoscope 2 includes an imaging optical system and an imaging device. The ultrasound endoscope 2 is inserted into a digestive tract (the esophagus, the stomach, the duodenum or the large intestine) or a respiratory organ (the trachea or a bronchi) of a subject and is able to capture images of the digestive tract or the respiratory organ. It is also possible to capture images of organs (the pancreas, the gallbladder, the bile duct, the duct of pancreas, lymph nodes, the organ in the mediastinum, blood vessels, etc.) around the digestive tract or the respiratory organ. The ultrasound endoscope 2 includes a light guide that guides the illumination light that is applied to the subject to capture an image optically. While the distal end of the light guide reaches the distal end of the part of the ultrasound endoscope 2 to be inserted into the subject, the proximal end of the light guide is connected to the light source device 6 that generates illumination light.

As illustrated in FIG. 1, the ultrasound endoscope 2 includes an insertion part 21, an operation unit 22, an universal code 23, and a connector 24. The insertion part 21 is a part that is inserted into the subject. As illustrated in FIG. 1, the insertion part 21 includes a distal-end rigid part 211, a curve part 212 that is joined to a proximal end side of the distal-end rigid part 211, and a flexible tube 213 that is joined to a proximal end side of the curve part 212 and that has flexibility. Although specific illustration in the drawings is omitted, a light guide that transmits illumination light that is supplied from the light source device 6 and a plurality of signal cables that transmit various signals are inserted into the insertion part 21 and a channel (treatment tool channel to be described below) that forms a treatment tool insertion path for inserting the treatment tool is inserted into the insertion part 21. The configuration of the distal end of the insertion part 21 will be described below.

The operation unit 22 is an unit that is joined to the proximal end side of the insertion part 21 and that receives various operations from a doctor, or the like. As illustrated in FIG. 1, the operation unit 22 includes a curve knob 221 for giving an operation to curve the curve part 212 and a plurality of operation members 222 for performing various operations. In the operation part 22, a treatment tool insertion port 223 for inserting a treatment tool into the treatment tool insertion path, which is a treatment tool insertion port communicating with the treatment tool channel, is formed.

The universal code 23 is a cable that extends from the operation unit 22 and in which a plurality of signal cables that transmit various signals and an optical fiber that transmits the illumination light supplied from the light source device 6, or the like, are provided.

The connector 24 is provided at the distal end of the universal code 23. The connector 24 includes first to third connectors 241 to 243 to which an ultrasound cable 31, a video cable 41, and the light source device 6 are connected, respectively.

The ultrasound observation apparatus 3 is electrically connected to the ultrasound endoscope 2 via the ultrasound cable 31 (refer to FIG. 1) and outputs a pulse signal to the ultrasound endoscope 2 via the ultrasound cable 31 and the echo signal is input to the ultrasound observation apparatus 3 from the ultrasound endoscope 2. The ultrasound observation apparatus 3 performs given processing on the echo signal to generate an ultrasound image.

The endoscope observation apparatus 4 is electrically connected to the ultrasound endoscope 2 via the video cable 41 (refer to FIG. 1) and an image signal from the ultrasound endoscope 2 is input to the endoscope observation apparatus 4 via the video cable 41. The endoscope observation apparatus 4 performs given processing on the image signal to generate an endoscopic image.

The display device 5 is formed using liquid crystals or electro luminescence (EL) and displays the ultrasound image that is generated by the ultrasound observation apparatus 3 or an endoscopic image that is generated by the endoscope observation apparatus 4.

The light source device 6 supplies the illumination light to the ultrasound endoscope 2 via an optical fiber cable 61.

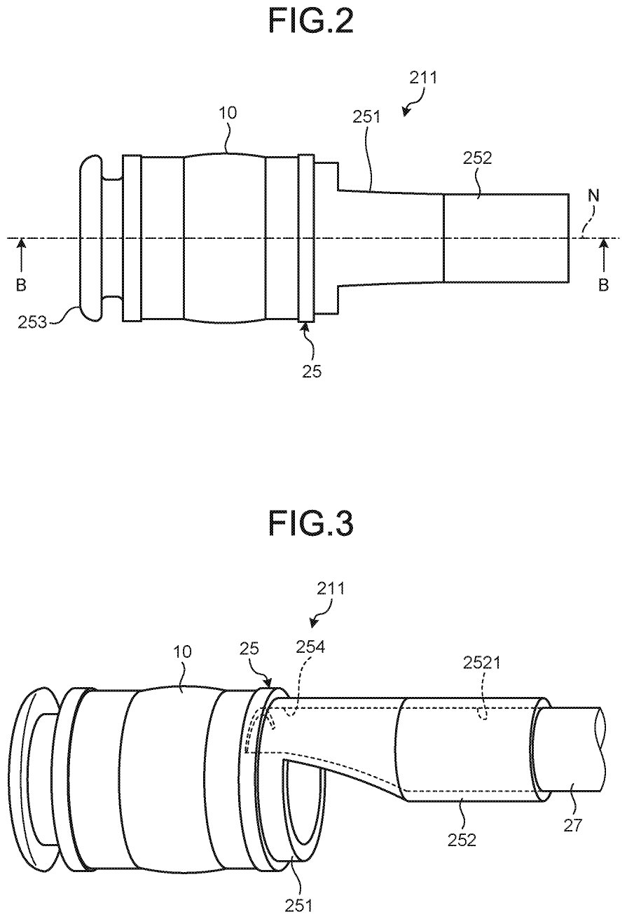

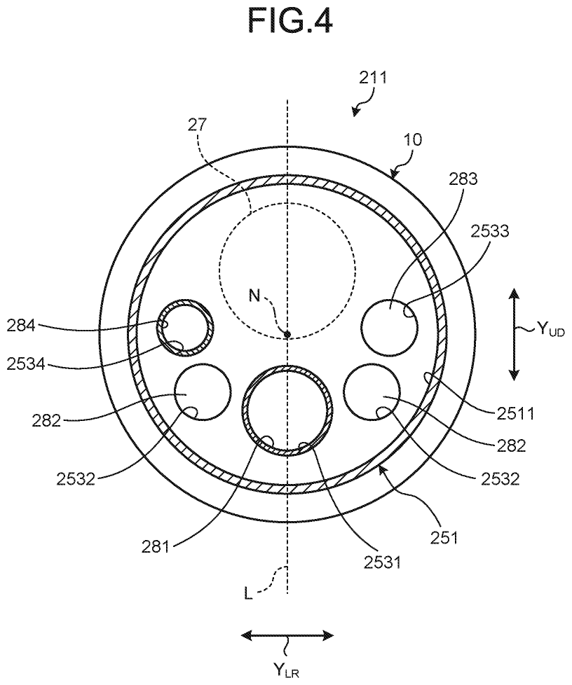

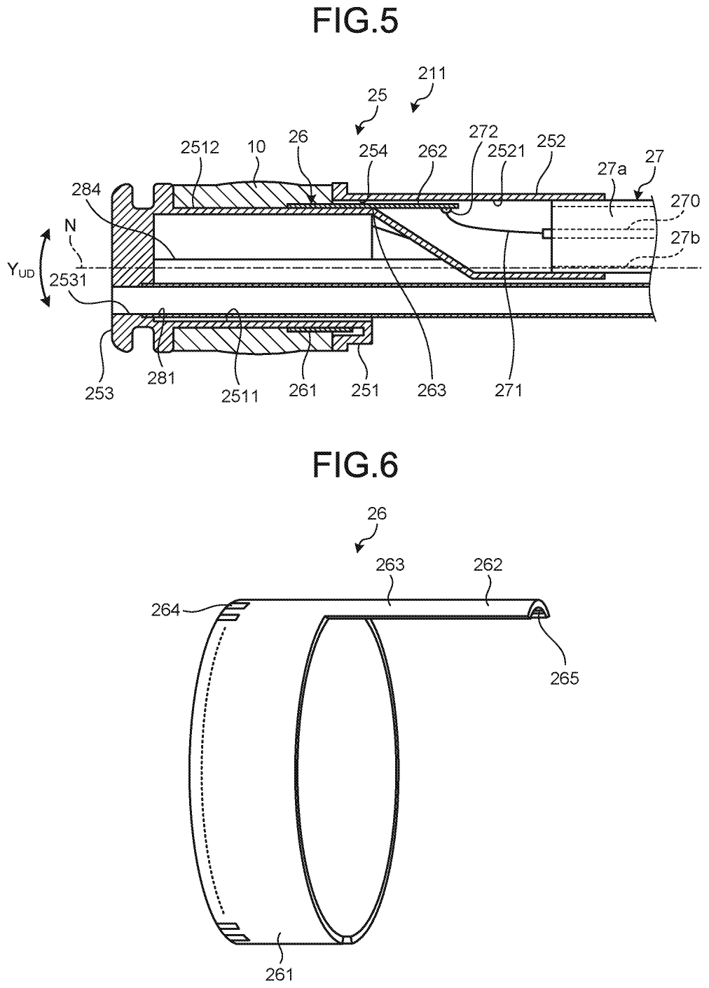

FIG. 2 is a side view schematically illustrating the configuration of the distal end of the insertion part of the ultrasound endoscope according to the first embodiment of the disclosure. FIG. 3 is a perspective view schematically illustrating the configuration of the distal end of the insertion part of the ultrasound endoscope according to the first embodiment of the disclosure. FIG. 4 is a cross-sectional view taken along the A-A line in FIG. 1. FIG. 5 is a cross-sectional view taken along the B-B line represented in FIG. 2. For description, FIGS. 2 and 3 illustrate the configuration of only an ultrasound transducer 10 and the distal-end rigid part 211.

The distal-end rigid part 211 includes a hard member 25 that is formed using a rigid material, a flexible substrate 26 that is at least partly provided in the hard member 25, and the above-described ultrasound transducer 10. The outer surface of the distal-end rigid part 211 is formed of the ultrasound transducer 10 and the hard member 25 and thus has rigidity. The hard member 25 includes a function part 251 that holds the ultrasound transducer 10 on a side part and a holder 252 that extends from the proximal end side of the function part 251 and holds an ultrasound cable 27 that is electrically connected to the ultrasound transducer 10 via the flexible substrate 26. In the hard member 25, a balloon engaging part with which an end and the other end of a balloon that can be filled with an ultrasound medium can be engaged is formed on each of the distal end side and the proximal end side with respect to the ultrasound transducer 10.

In the function part 251, a first hole 2511, a concave part 2512 that serves as part of the outer circumferential surface of the function part 251 and to which the ultrasound transducer 10 is attached, and holder holes 2531 to 2534 each of which communicates with the first hole 2511 are formed. In the function part 251, a treatment tool channel 281 that communicates with the treatment tool insertion path formed in the insertion part 21, that causes the treatment tool to protrude from the distal end of the insertion part 21, and sucks a fluid, such as the liquid or gas in the subject; a light guide 282 that guides the illumination light; a forward viewing optical unit 283 that is formed of at least one lens, an imaging sensor, etc., and on which observation light for generating an internal forward viewing image of the subject; and a gas transmission liquid transmission tube 284 that has a nozzle arranged at its distal end and that sends a fluid, such as liquid and gas, into the subject are provided. Through the first hole 2511, the treatment tool channel 281, the light guide 282, the gas transmission liquid transmission tube 284, and a cable that is connected to the imaging device of the forward viewing optical unit 283 penetrate. The forward viewing optical unit 283 corresponds to an imaging unit.

As illustrated in FIG. 4, in the hard member 25, the holder hole 2531 that holds an end of the treatment tool channel 281, the holder hole 2532 that holds an end of the light guide 282, the holder hole 2533 that holds an optical member that is positioned at the distal end of the forward viewing optical unit 283, and the holder hole 2534 that holds an end of the gas transmission liquid transmission tube 284 are formed. The treatment tool channel 281, the light guide 282, the forward viewing optical unit 283, and the gas transmission liquid transmission tube 284 are held by the holder holes 2531 to 2534, respectively, in a watertight manner. The treatment tool channel 281 has an opening at its distal end in its longitudinal direction and the opening communicates with the holder hole 2531.

On the other hand, in the holder 252, a second hole 2521 that can hold the ultrasound cable 27 is formed. The second hole 2521 forms a hole shape that extends with its diameter gradually increasing from the distal end side to the proximal end side and then being kept uniform. The maximum diameter of the outer diameter of the holder 252 is smaller than the diameter of the first hole 2511 of the function part 251.

In the hard member 25, the first concave part 2512 of the function part 251 and the second hole 2521 of the holder 252 communicate with each other via a communication part 254.

The ultrasound transducer 10 is a radial transducer that performs scanning by applying ultrasound in a direction orthogonal to the longitudinal direction of the insertion part 21 (for example, the direction of the center axis N of the distal-end rigid part 211) to positions around an axis parallel with the longitudinal direction. The ultrasound transducer 10 includes a plurality of piezoelectric elements that are arranged along the circumferential direction of the ultrasound transducer 10 and performs electric scanning by electrically switching among the piezoelectric elements relating to transmission and reception and delaying transmission and reception of each of the piezoelectric elements. The piezoelectric elements vibrate in response to input of the pulse signal and accordingly the ultrasound transducer 10 applies ultrasound to the object to be observed. The ultrasound that is reflected from the object to be observed is transmitted to the piezoelectric elements. The transmitted ultrasound causes the piezoelectric elements to vibrate and the piezoelectric elements convert the vibration into an electric signal and outputs the electric signal as the echo signal to the ultrasound observation apparatus 3 via the flexible substrate 26 and the ultrasound cable 27, etc.

The ultrasound transducer 10 applies ultrasound in the circumferential direction by causing each of the piezoelectric elements to vibrate sequentially and receives ultrasound echoes that are reflected by the object to be observed. In other words, the ultrasound transducer 10 receives the ultrasound echoes that form a cross-sectional image of the annular scanned surface around the ultrasound transducer 10. On the outer surface of the ultrasound transducer 10, the central part of the ultrasound transducer 10 along the longitudinal direction of the insertion part 21 more protrudes in the direction orthogonal to the longitudinal direction than both ends of the ultrasound transducer 10 in the longitudinal direction do. For example, an acoustic lens forms the outer surface of the ultrasound transducer 10. The acoustic lens has a convex shape toward the central part and thus has a function of narrowing ultrasound and emits the ultrasound transmitted by the piezoelectric elements to the outside or takes in ultrasound echoes from the outside. In the first embodiment, the acoustic lens of the ultrasound transducer 10 will be described as one having a convex shape in the case where a material in which the acoustic velocity is lower than that in the object to be observed, such as silicone, is used. Alternatively, an acoustic lens material in which the acoustic velocity is higher than that in the object to be observed may be used such that the acoustic lens has a concave shape.

The ultrasound transducer 10 is connected to the flexible substrate 26. One end side of the flexible substrate 26 in the direction of the center axis N is connected to the ultrasound transducer 10 and the other end side enters the second hole 2521 of the holder 252 via the communication part 254. On one side of the flexible substrate 26, electrodes connected to the respective piezoelectric elements of the ultrasound transducer 10 and the interconnection pattern that is formed on the flexible substrate 26 are fixed with a conductive fixing member, such as solder. On the other side, the flexible substrate 26 is connected to the ultrasound cable 27 in the second hole 2521.

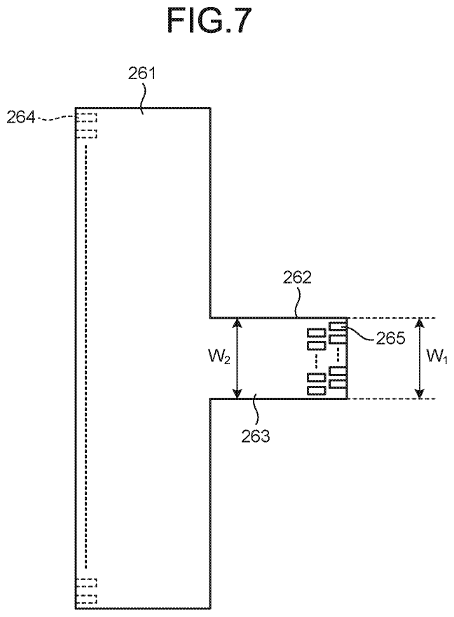

FIG. 6 is a schematic view illustrating a configuration of the flexible substrate of the ultrasound endoscope according to the first embodiment of the disclosure. FIG. 7 is a developed view of the flexible substrate illustrated in FIG. 6. As illustrated in FIG. 6, the flexible substrate 26 includes a first connection part 261 that is connected to the ultrasound transducer 10, a second connection part 262 that is connected to each core 271 of the ultrasound cable 27, and a joint 263 that joins a central part of the first connection part 261 in its circumferential direction with the second connection part 262.

The first connection part 261 curves such that the identical main surfaces face with each other, forming an annular shape partly disconnected in the circumferential direction. In the first connection part 261, electrodes (a plurality of electrodes 264 in FIG. 7) that are connected to the respective electrodes of the ultrasound transducer 10 are formed along the circumferential direction. The main surfaces denote surfaces having the largest areas.

The second connection part 262 curves to the same side as that to which the first connection part 261 curves. In the second connection part 262, electrodes (electrodes 265 in FIG. 7) each of which is connected to any one of the electrodes (the electrodes 264) that are formed in the first connection part 261 by the interconnection pattern (not illustrated in the drawings) and is connected to the core (the core 271) of the ultrasound cable 27 are formed.

Through the joint 263, the interconnection pattern passes. The joint 263 being provided in the hard member 25 (refer to FIG. 5) penetrates through the communication part 254.

In the flexible substrate 26, when the length in the circumferential direction is set for the width, the width of the second connection part 262 (a width w₁ in FIG. 7) and the width of the joint 263 (a width w₂ in FIG. 7) are equal to each other.

The ultrasound cable 27 is formed by covering a plurality of coaxial cores 270 that are provided according to the number of piezoelectric elements to which the coaxial cores 270 are connected with an insulating jacket 27a. The jacket 27a covers the coaxial cores 270 that are bundled. An integration shield 27b is provided on the inner circumference of the jacket 27a. The dashed circle in FIG. 4 represents the outer diameter of the jacket 27a. The coaxial cores 270 are formed of a conductive core (a core 271), a dielectric layer (not illustrated in the drawings) that covers the core 271, a shield (not illustrated in the drawings) that covers the dielectric layer, and an insulative protective coating (not illustrated in the drawings) that covers the shield. In FIG. 5, only the core 271 that is one of the cores extends for explanation; however, practically, there are cores (the coaxial cores 270) corresponding to the number of piezoelectric elements to which the cores are connected.

The ultrasound cable 27 is held by the holder 252 with the jacket 27a being inserted from the proximal end side of the holder 252. The jacket 27a is press fitted to the holder 252 or is fixed to the second hole 2521 of the holder 252 with an adhesive material, or the like. Each of the coaxial cores 270 being covered with the jacket 27a to the holder 252 is inserted into the insertion part 21 and the core 271 is exposed at the second hole 2521 of the holder 252. In other words, each of the coaxial cores 270 is covered with the jacket 27a until the coaxial core reaches the proximal end side of the distal-end rigid part 211 from the flexible tube 213 via the curve part 212 and is fixed to the holder 252 with insulation being kept.

The holder 252 is positioned on the side of the outer circumference of the function part 251. For this reason, the ultrasound cable 27 held by the holder 252 is also positioned on the side of the outer circumference of the function part 251. In other words, in the first embodiment, the ultrasound cable 27 is positioned on the side of the outer circumference of the hard member 25 in the radial direction orthogonal to the direction of the center axis N (refer to FIG. 4). In the first embodiment, in a cross-section passing through the end of the jacket 27a in the distal-end rigid part 211, a straight line L passing through the center axis N and orthogonal to the canter axis N passes through the center of the ultrasound cable 27 and the center of the channel 281. In the hard member 25, the ultrasound cable 27 and the channel 281 have diameters larger than those of other contents. For this reason, arranging the ultrasound cable 27 and the channel 281 along the straight line L passing through the center axis N and being orthogonal to the center axis N enables minimization of the diameter of the distal-end rigid part 211. Furthermore, the straight line L is parallel with a curve direction Y_{UD} (refer to FIGS. 4 and 5) that is a direction of curve of the curve part 212 and that corresponds to the vertical direction of an image to be captured, which makes it possible to, when the curve part 212 is curved in the direction Y_{UD}, reduce shakes in a horizontal direction Y_{LR} orthogonal to the curve direction Y_{UD}.

In the above-described first embodiment, the ultrasound cable 27 formed by covering the coaxial cores 270 with the insulative jacket 27a is connected at the insulative hard member 25 that is positioned at the distal end of the curve part 212 and the core 271 is exposed at the hard member 25 and is connected to the flexible substrate 26. According to the first embodiment, the ultrasound cable 27 is inserted into the curve part 212 with the coaxial cores being bundled with the jacket 27a and the integration shield 27b that is arranged on the inner side of the jacket 27a, which makes it possible to reduce noise that is superimposed onto the coaxial cores and noise that is emitted from the coaxial cores. The ultrasound cable 27 obtained by bundling the coaxial cores 270 passes through the curve part 212 and accordingly the area occupied by the ultrasound cable 27 in the insertion part 21 is smaller than that in the case where a flexible substrate is used, which inhibits an increase in diameter. Accordingly, in the configuration including the radial ultrasound transducer 10, the forward viewing optical unit 283, and the channel 281, it is possible to reduce noise and inhibit an increase in diameter of the insertion part. On the other hand, in a conventional configuration where coaxial cores and a flexible substrate are connected to each other on the proximal end side of the curve part and the flexible substrate is inserted into the curve part, noise tends to give effects and, in order to keep resistance to noise, it is necessary to increase the thickness of the flexible substrate and thus it is difficult to reduce the diameter.

According to the above-described first embodiment, the holder 252 and the ultrasound cable 27 are connectable to each other by press fitting or an adhesive, which makes it possible to easily connect the ultrasound cable 27 to the hard member 25 while maintaining insulation.

According to the above-described first embodiment, the ultrasound cable 27 is arranged in the curve part 212 with the coaxial cores 270 being covered with the jacket 27a and thus disconnection of the coaxial cores does not tend to occur. Keeping the withstand voltage performance of the coaxial cores 270 using the jacket 27a covering the coaxial cores 270 and the holder 252 that holds the jacket 27a achieves an ultrasound endoscope that is highly safe electrically.

In the above-described first embodiment, the ultrasound cable 27 is inserted with the coaxial cores bundled by being covered with the jacket 27a from the flexible tube 213 to the curve part 212 and accordingly the ultrasound cable 27 does not tend to tangle with other contents, which enables improvement in operability in fixing.

### Second Embodiment

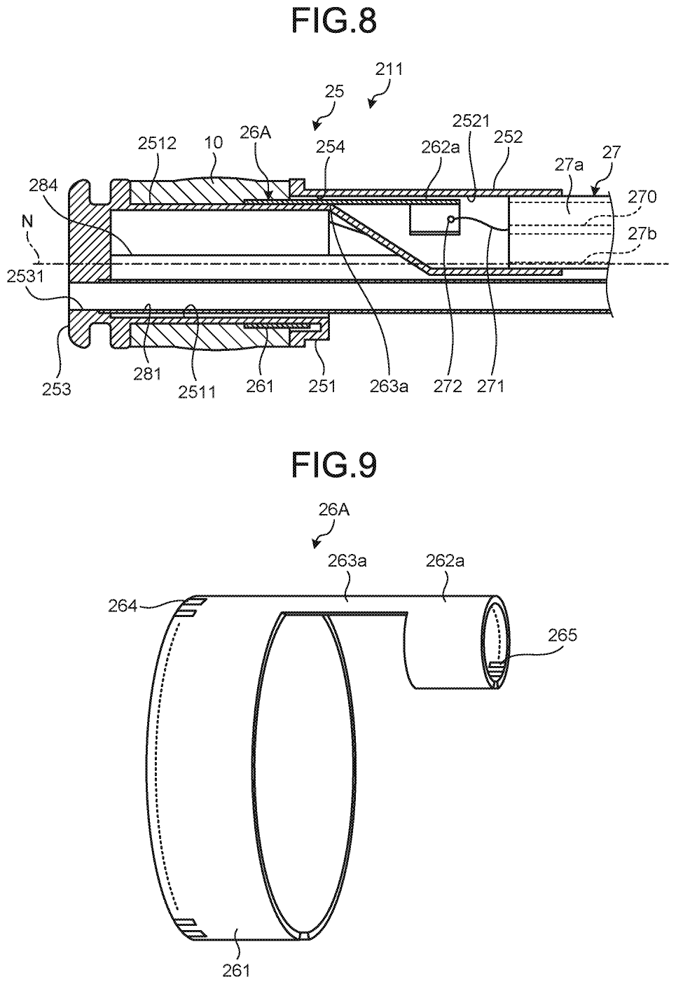

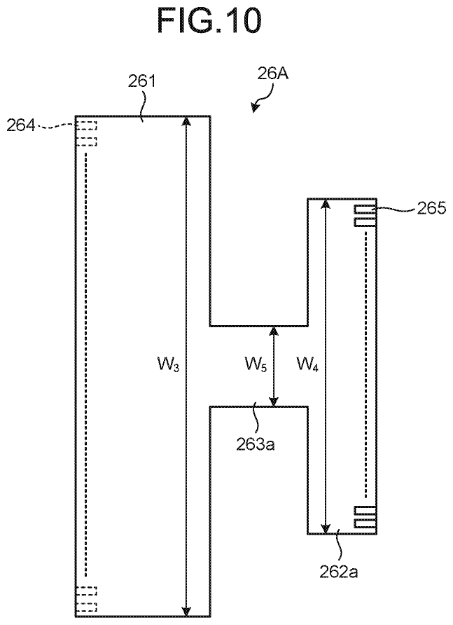

FIG. 8 is a cross-sectional view schematically illustrating a configuration of a distal end of an insertion part of an ultrasound endoscope according to a second embodiment of the disclosure. FIG. 9 is a schematic view illustrating a configuration of a flexible substrate of the ultrasound endoscope according to the second embodiment of the disclosure. FIG. 10 is a developed view of the flexible substrate illustrated in FIG. 9.

The distal-end rigid part 211 of the ultrasound endoscope 2 according to the second embodiment includes a flexible substrate 26A instead of the flexible substrate 26 of the above-described first embodiment (refer to FIG. 2). The distal-end rigid part 211 has the same configuration as that of the above-described first embodiment except for the change of the flexible substrate. The flexible substrate 26A includes the first connection part 261 that forms an annular shape partly disconnected in the circumferential direction and that is connected to the ultrasound transducer 10, a second connection part 262a that forms an annular shape partly disconnected in the circumferential direction and that is connected to each core 271 of the ultrasound cable 27, and a joint 263a that joins the central parts of the first connection part 261 and the second connection part 262a in the circumferential direction.

In the second connection part 262a, electrodes (the electrodes 265 in FIG. 10) each of which is connected to any one of the electrodes that are formed in the first connection part 261 with an interconnection pattern (not illustrated in the drawings) and is connected to the core of the ultrasound cable 27 (the core 271 illustrated in FIG. 8) are formed. Although ground lines of the respective coaxial cores 270 are not illustrated in the drawings, the ground lines are gathered near an end of the jacket 27a on the proximal end side and are connected to the electrodes on the ground side (outer circumferential surface side) of the piezoelectric elements via a dedicated pattern that is provided in the flexible substrate 26A or via a connection cable that is provided independently.

The above-described interconnection pattern passes through the joint 263a. The joint 263a being provided in the hard member 25 penetrates through the communication part 254.

In the flexible substrate 26A, a width of the first connection part 261 (a width w₃ in FIG. 10), a width of the second connection part 262a (a width w₄ in FIG. 10), and a width of the joint 263a (a width w₅ in FIG. 10) have a relationship of w₅<w₃ and w₅<w₄.

In the above-described second embodiment, the ultrasound transducer 10 and the ultrasound cable 27 are electrically connected to each other using the flexible substrate 26A where the width of the first connection part 261 (the width w₃ in FIG. 10), the width of the second connection part 262a (the width w₄ in FIG. 10), and the width of the joint 263a (the width w₅ in FIG. 10) have the relationship of w₅<w₄<w₃. This makes it possible to increase the distance between the electrodes 265 adjacent to each other in the circumferential direction compared to the second connection part 262 of the flexible substrate 26 according to the above-described first embodiment. As a result, it is possible to inhibit interference between the adjacent cores 271 with more certainty and improve operability in connecting the cores 271 with the electrodes 265 during manufacturing.

The second connection part 262a that is connected to each of the cores 271 of the ultrasound cable 27 need not be annular, and the second connection part 262a may be spiral or folded. Forming the second connection part 262a that is spiral makes it possible to increase the width w₄ enabling connection to the cable. Increasing the width w₄ enabling connection to the cable such that w₅<w₃<w₄ is satisfied and keeping the pitch between the electrodes 265 on the cable side equal to or larger than the thickness of the cores of the cable enable easy positioning of the flexible substrate and the cable and easy wiring operations.

### Modification 1 of Second Embodiment

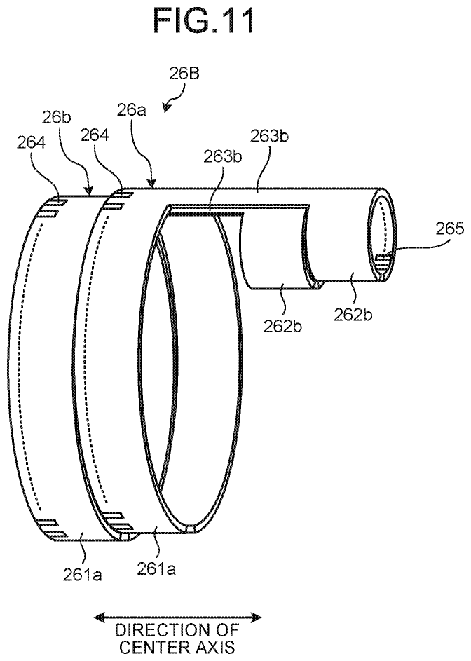

FIG. 11 is a schematic view illustrating a configuration of a flexible substrate of an ultrasound endoscope according to Modification 1 of the second embodiment of the disclosure.

A flexible substrate 26B according to Modification 1 is formed by partly overlapping two flexible substrates (a first flexible substrate 26a and a second flexible substrate 26b).

The first flexible substrate 26a includes a first connection part 261a that forms an annular shape partly disconnected in its circumferential direction and that is connected to the ultrasound transducer 10; a second connection part 262b that forms an annular shape partly disconnected in its circumferential direction and that is connected to each of the cores 271 of the ultrasound cable 27; and a joint 263b that joins central parts of the first connection part 261a and the second connection part 262b in the circumferential direction.

The second flexible substrate 26b has the same configuration as that of the first flexible substrate 26a. The second flexible substrate 26b includes the first connection part 261a, the second connection part 262b, and the joint 263b.

In the first connection part 261a, the electrodes 264 to be connected to the respective electrodes of the ultrasound transducer 10 are formed along the circumferential direction. The electrodes 264 that are connected to the respective piezoelectric elements of the ultrasound transducer 10 are formed in each of the connection parts 261a of the first flexible substrate 26a and the second flexible substrate 26b.

In each of the second connection parts 262b of the first flexible substrate 26a and the second flexible substrate 26b, the electrodes 265 that are electrodes each of which is connected to any one of the electrodes formed in the first connection part 261a with an interconnection pattern (not illustrated in the drawings) and that are connected to the cores (the cores 271 illustrated in FIG. 8) of the ultrasound cable 27 are formed.

In Modification 1, all the electrodes 264 that are formed in the first connection part 261 according to the second embodiment are provided separately in each of the first connection parts 261a of the first flexible substrate 26a and the second flexible substrate 26b. All the electrodes 264 that are formed in the second connection part 262a according to the second embodiment are provided separately in each of the second connection parts 262b of the first flexible substrate 26a and the second flexible substrate 26b. This keeps the number of the electrodes 264 and the number of the electrodes 265 that are formed in each of the first connection parts 261a and each of the second connection parts 262b at approximately a half of the numbers of electrodes that are formed in the first connection part 261a and the second connection part 262a according to the above-described second embodiment.

The aforementioned interconnection pattern passes through the joint 263b.

The width of the first connection part 261a, the width of the second connection part 262b, and the width of the joint 263b have the same relationship (w₅<w₄<w₃) as that of the widths of the above-described second embodiment.

The first flexible substrate 26a and the second flexible substrate 26b are provided in the hard member 25 such that the first connection part 261a and the second connection part 262b are adjacent with each other in the direction of the center axis N and the joints 263b and 263b overlap partly. The joints 263b and 263b penetrate through the communication part 254.

In Modification 1 described above, the flexible substrate 26B formed by overlapping the first flexible substrate 26a and the second flexible substrate 26b is formed. This makes it possible to halve the number of the electrodes 264 and the number of electrodes 265 that are formed in one of the first connection parts 261a and one of the second connection parts 262b. As a result, it is possible to reduce the interconnection density of the interconnection pattern that is formed in the first flexible substrate 26a and the second flexible substrate 26b and thus deal with an increase in the number of elements.

Furthermore, according to Modification 1, the joints 263b being overlapping with each other penetrate through the communication part 254, which makes it possible to provide the joints 263b in the hard member 25 without reducing the width of the joint of each of the flexible substrates. As a result, even when multiple substrates are used, it is possible to reduce the interconnection density in the joints 263b and increase the line width of the interconnection. Increasing the line width of the interconnection can inhibit the wiring resistance from increasing.

In Modification 1, the second connection parts 262b of the first flexible substrate 26a and the second flexible substrate 26b have been described as ones adjacent with each other in the direction of the center axis N. Alternatively, the second connection parts 262b may overlap partly.

### Modification 2 of Second Embodiment

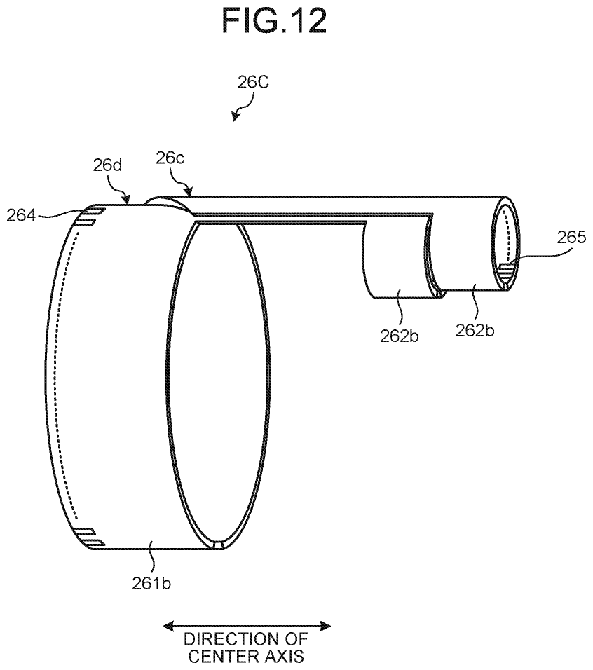

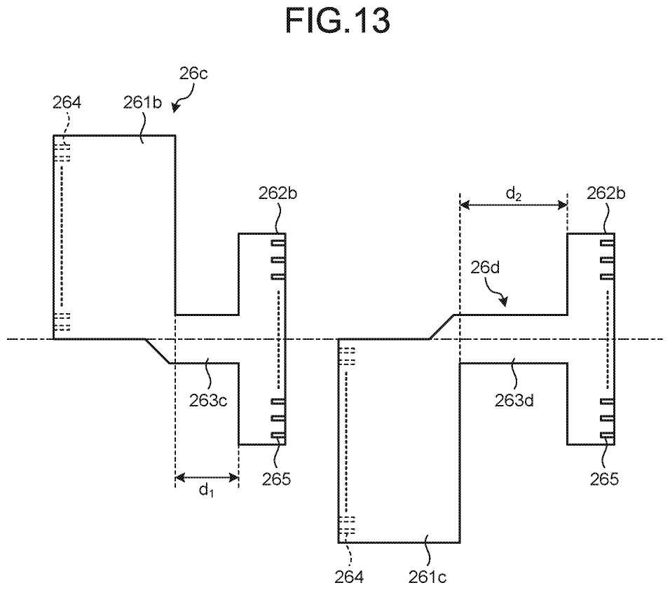

FIG. 12 is a schematic view illustrating a configuration of a flexible substrate of an ultrasound endoscope according to Modification 2 of the second embodiment of the disclosure. FIG. 13 is a developed view of the flexible substrate illustrated in FIG. 12.

A flexible substrate 26C according to Modification 2 is formed by partly overlapping two flexible substrates (a first flexible substrate 26c and a second flexible substrate 26d).

The first flexible substrate 26c includes a first connection part 261b that extends and forms an arc and that is connected to the ultrasound transducer 10, a second connection part 262b that forms an annular shape disconnected partly in its circumferential direction and that is connected to each of the cores 271 of the ultrasound cable 27, and a joint 263c that joins one end of the first connection part 261b and a central part of the second connection part 262b in the circumferential direction.

The aforementioned interconnection pattern passes through the joint 263c. The joint 263c being provided in the hard member 25 penetrates through the communication part 254.

The second flexible substrate 26d includes a first connection part 261c that extends and forms an arc that curves in a direction opposite to that of the first connection part 261b and that is connected to the ultrasound transducer 10; a second connection part 262b that forms an annular shape disconnected partly in its circumferential direction and that is connected to each of the cores 271 of the ultrasound cable 271; and the joint 263d that joins one end of the first connection part 261c in the circumferential direction and a central part of the second connection part 262b in the circumferential direction.

The aforementioned interconnection pattern passes through the joint 263d. The joint 263c being provided in the hard member 25 penetrates through the communication part 254. A length d₂ between the first connection part 261c and the second connection part 262b achieved by the joining by the joint 263d in the second flexible substrate 26d is larger than a length d₁ between the first connection part 261b and the second connection part 262b achieved by the joining by the joint 263c in the first flexible substrate 26c.

The first flexible substrate 26c and the second flexible substrate 26d are provided in the hard member 25 with their second connection parts 262b being adjacent to each other and with the joints 263b overlapping partly. The overlapping joints 263c and 263d penetrate through the communication part 254. The first connection parts 261b and 261c extend in opposite directions and form arcs, thereby forming an intermittent cylinder having an inner diameter along the surface of the concave part 2512.

As the above-described Modification 1 does, Modification 2 described above makes it possible to reduce the interconnection density of the interconnection pattern and deal with an increase in the number of elements.

According to Modification 2, it is possible to connect the piezoelectric elements to the two flexible substrates 26c and 26d at the proximal end side end of the piezoelectric elements, which makes it easier to connect the piezoelectric elements and the flexible substrates.

According to Modification 2, as described in the second embodiment, increasing the width of the second connection part 262b more than that of the first connection part 261b enables a pitch between the electrodes 265 that are connected to the cable to be equal to or larger than the thickness of the cores of the cable, thus enabling easy positioning of the flexible substrates and the cable and easy wiring operations.

### Third Embodiment

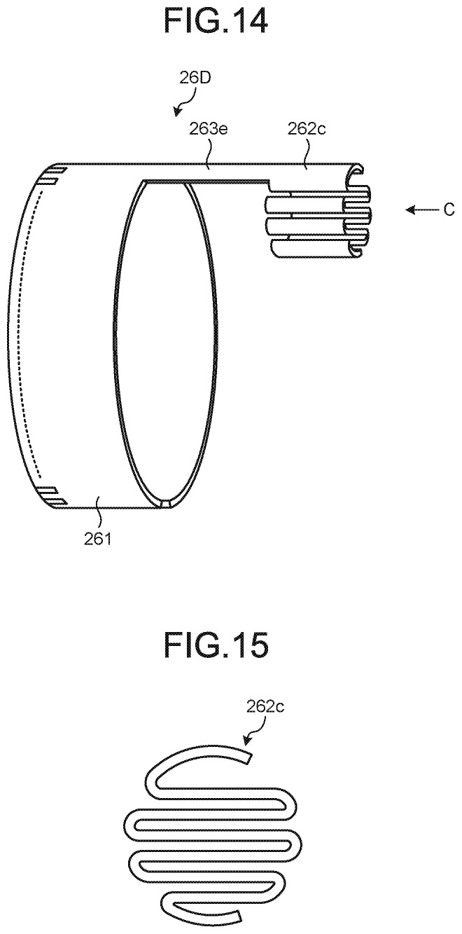

FIG. 14 is a schematic view illustrating a configuration of a flexible substrate of an ultrasound endoscope according to a third embodiment of the disclosure. FIG. 15 is a plane view in a direction of the arrow C in FIG. 14 and is a plane view illustrating a configuration of a second connection part 262c.

The distal-end rigid part 211 of the ultrasound endoscope 2 according to the third embodiment includes a flexible substrate 26D instead of the flexible substrate 26 having the configuration (see FIG. 2) of the above-described first embodiment. The distal-end rigid part 211 has the same configuration as that of the above-described first embodiment except for the change of the flexible substrate. The flexible substrate 26D includes the first connection part 261 that forms an annular shape partly disconnected in its circumferential direction and that is connected to the ultrasound transducer 10, the second connection part 262c that forms an annular shape partly disconnected in its circumferential direction and that is connected to each of the cores 271 of the ultrasound cable 27, and a joint 263e that joins the first connection part 261 and the second connection part 262c.

The second connection part 262c forms a zigzag whose direction of extension inverses along its longitudinal direction. In the second connection part 262c, electrodes (for example, the electrodes 265) each of which is an electrode that is connected to any one of the electrodes that are formed in the first connection part 261 with an interconnection pattern (not illustrated in the drawings) and that is connected to the core 271 of the ultrasound cable 27 are formed. In the direction of the arrow C in FIG. 14, a circle circumscribed by the second connection part 262c has an inner diameter smaller than that of the second hole 2521. In the third embodiment 3, the longitudinal direction of a belt-like component forming the second connection part 262c is orthogonal to the direction of the aforementioned center axis N. The second connection part 262c may be formed using a belt-like component forming a zig-zag along the longitudinal direction parallel with the aforementioned direction of the center axis N.

The above-described interconnection pattern passes through the joint 263e. The joint 263e being provide in the hard member 25 penetrates through the communication part 254.

In the third embodiment described above, the ultrasound transducer 10 and the ultrasound cable 27 are electrically connected to each other using the flexible substrate 26D including the second connection part 262c forming a zig-zag. This makes it possible to increase the distance between the electrodes 265 adjacent to each other compared to the second connection part 262 of the flexible substrate 26 according to the above-described first embodiment. As a result, it is possible to inhibit interference between the adjacent cores 271 with more certainty.

### Fourth Embodiment

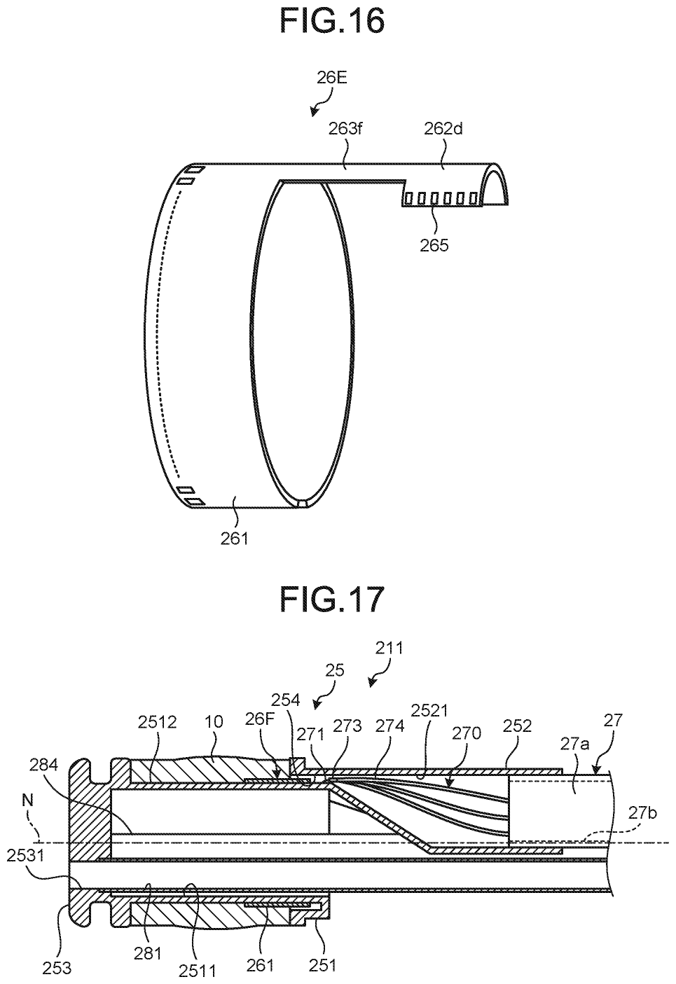

FIG. 16 is a schematic view illustrating a configuration of a flexible substrate of an ultrasound endoscope according to a fourth embodiment of the disclosure.

The distal-end rigid part 211 of the ultrasound endoscope 2 according to the fourth embodiment includes a flexible substrate 26E instead of the flexible substrate 26 having the configuration of the above-described first embodiment (refer to FIG. 2). The distal-end rigid part 211 has the same configuration as that of the above-described first embodiment except for the change of the flexible substrate. The flexible substrate 26E includes the first connection part 261 that forms an annular shape partly disconnected in its circumferential direction and that is connected to the ultrasound transducer 10, a second connection part 262d that forms an annular shape partly disconnected in its circumferential direction and that is connected to each of the cores 271 of the ultrasound cable 27, and a joint 263f that joins central parts of the first connection part 261 and the second connection part 262d in the circumferential direction.

In the second connection part 262d, the electrodes 265 each of which is an electrode that is connected to any one of the electrodes that are formed in the first connection part 261 with an interconnection pattern (not illustrated in the drawings) and that is connected to the core 271 of the ultrasound cable 27 are formed. The electrodes 265 are formed on the same side as a side on which the electrodes 264 of the first connection part 261 are formed, and the longitudinal direction is parallel with the width direction. Each of the electrodes 265 is formed along the direction orthogonal to the width direction on the surface of the second connection part 262d. The second connection part 262d has a width larger than that of the joint 263f.

The above-described interconnection pattern passes through the joint 263f. The joint 263f being provided in the hard member 25 penetrates through the communication part 254.

In the fourth embodiment described above, each of the electrodes 265 is formed along the direction orthogonal to the width direction on the surface of the second connection part 262d. In such a configuration, it is possible to reduce the interconnection density of the interconnection pattern described above and deal with an increase in the number of elements.

In the above-described fourth embodiment, the electrodes 265 are described as ones whose longitudinal direction is parallel with the width direction. The longitudinal direction may be parallel with the width direction or the longitudinal direction may be oblique to the width direction (for example, forming an acute angle).

### Fifth Embodiment

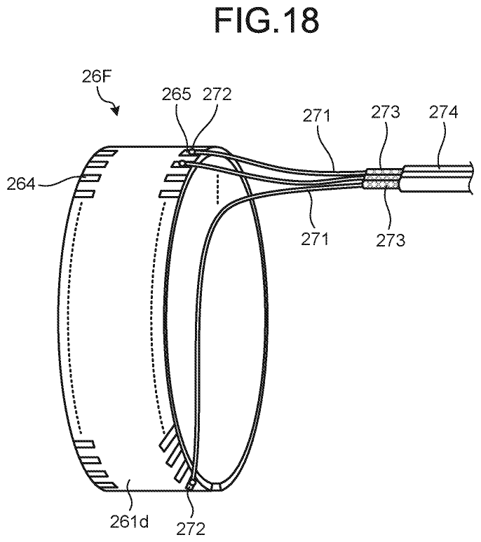

FIG. 17 is a cross-sectional view schematically illustrating a configuration of a distal end of an insertion part of an ultrasound endoscope according to a fifth embodiment of the disclosure. FIG. 18 is a schematic view illustrating a mode of connection between a flexible substrate and a cable of the ultrasound endoscope according to the fifth embodiment of the disclosure.

The distal-end rigid part 211 of the ultrasound endoscope 2 according to the fifth embodiment includes a flexible substrate 26F instead of the flexible substrate 26 having the configuration of the above-described first embodiment (refer to FIG. 2). In the above-described first to fourth embodiments, the joint that joins the first connection part and the second connection part on the flexible substrate is described as one that penetrates through the communication part. In the fifth embodiment, the part of the coaxial cores penetrate through the communication part 254.

The flexible substrate 26F is formed of a main part 261d that forms an annular shape partly disconnected in its circumferential direction, that is connected to the ultrasound transducer 10 on one end side, and that is connected to each of the cores 271 of the ultrasound cable 27 on the other end side.

The main part 261d forms an annular shape partly disconnected in the circumferential direction. In the main part 261d, the electrodes 264 that are connected to the respective electrodes of the ultrasound transducer 10 are formed along the circumferential direction and the electrodes 265 each of which is an electrode that is connected to any one of the electrodes that are formed in the first connection part 261 by an interconnection pattern (not illustrated in the drawings) and that is connected to the core 271 of the ultrasound cable 27 are formed along the circumferential direction.

The coaxial cores 270 extend from the second hole 2521 to the communication part 254 with the cores 271 being covered with a protective film 274 and, after the coaxial cores 270 pass through the communication part 254, a shield 273 is exposed. The coaxial cores 270 are circumferentially along the flexible substrate 26F with the cores 271 (or an insulating layer) being exposed in the concave part 2512 and are connected to the electrodes to which the coaxial cores 271 are to be connected with a solder 272.

The electrodes 265 are oblique to the center axis N as the electrodes 265 separate from the communication part 254. In other words, the longitudinal direction of the electrodes 265 on a surface of connection is oblique to the direction in which the cores 271 of the coaxial cores 270 to be connected enter.

In the above-described firth embodiment, the coaxial cores 270 penetrate through the communication part 254 and the electrodes 265 oblique along the direction in which the cores 271 enter are connected to the cores 271. In such a configuration, it is possible to, as described above, reduce the interconnection density of the interconnection pattern and deal with an increase in the number of elements. The longitudinal directions of the electrodes 265 are aligned with the cores 271 and this enables reduction in the stress applied to the cores 271 that are connected to the electrodes 265.

In the above-described fifth embodiment, the coaxial cores 270 and the piezoelectric elements may be connected directly not via the flexible substrate 26F.

In the above-described fifth embodiment, the electrodes 265 have been described as ones whose longitudinal direction is parallel with the width direction. Alternatively, the longitudinal direction may be parallel with the width direction or the longitudinal direction may be oblique to the width direction (for example, oblique such that the longitudinal direction and the width direction form an acute angle).

The modes for carrying the disclosure has been described; however, the present disclosure is not limited by only the above-described embodiments and modifications. The disclosure is not limited to the above-described embodiments and medications and covers various embodiments within the scope of the technical idea described in claims. The configurations of the embodiments and the modifications may be combined as appropriate.

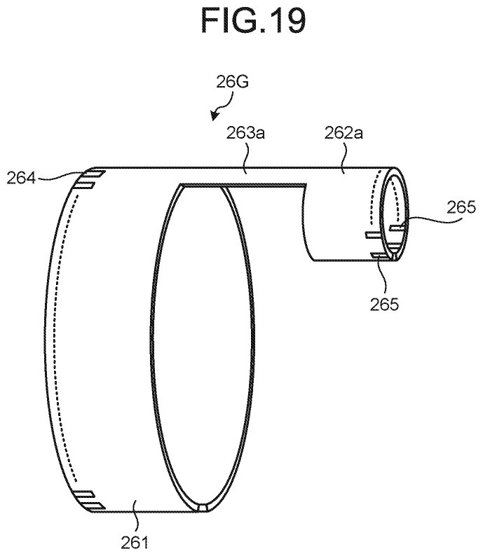

In the first to fifth embodiments, the example where the electrodes 264 and 265 are provided on a surface on one side of the flexible substrate has been described; however, the surface on which the electrodes are formed may be the surface on the opposite side and, for example, as illustrated in a flexible substrate 26G illustrated in FIG. 19, the electrodes 265 may be formed on both surfaces. The example where the electrodes are arranged in line along the circumferential direction has been described. Alternatively, the electrodes may be arranged in a plurality of lines along the circumferential direction.

As described above, the configuration of the ultrasound endoscope according to the disclosure including the radial ultrasound transducer, the forward viewing optical system, and the channel is useful to reduce noise and inhibit an increase in diameter of the insertion part.

According to the disclosure, an effect that, in a configuration including a radial ultrasound transducer, a forward viewing optical system and a channel, it is possible to reduce noise and inhibit an increase in diameter of an insertion part is achieved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound endoscope comprising:
an insertion part including
a distal-end rigid part has rigidity,
a curve part that is joined to a proximal end side of the distal-end rigid part and that can be curved in at least one direction, and
a flexible tube part that is joined to a proximal end side of the curve part and that has flexibility;
an ultrasound transducer in which a plurality of piezoelectric elements capable of transmitting and receiving ultrasound are arranged annularly along a circumferential direction of the distal-end rigid part, the ultrasound transducer being configured to apply the ultrasound in a direction orthogonal to a longitudinal direction of the insertion part;
an imaging sensor that is provided in the distal-end rigid part, the imaging sensor being configured to capture an forward-viewing image in the longitudinal direction of the insertion part;
an ultrasound cable including a plurality of coaxial cores that are electrically connected to the piezoelectric elements, respectively, and a metallic integration shield that covers the coaxial cores, the ultrasound cable being fixed to a proximal end side of the distal-end rigid part and on a side of an outer circumference; and
a flexible substrate configured to electrically connect the piezoelectric elements and the coaxial cores to each other,
wherein the flexible substrate includes
a first connection part that curves annularly and that is electrically connected to the piezoelectric elements,
a second connection part that forms an annular shape that curves to a same side as a side to which the first connection part curves and that is electrically connected to the first connection part and the coaxial cores, and
a joint configured to join the first connection part and the second connection part, and
the joint has a length of extension along a circumferential direction, which is a length smaller than lengths of extension of the first connection part and the second connection part in a circumferential direction.

2. The ultrasound endoscope according to claim 1, wherein the ultrasound cable further includes an insulative jacket that covers the integration shield, and
the jacket covering the coaxial cores reaches the proximal end side of the distal-end rigid part from the flexible tube part via the curve part.

3. The ultrasound endoscope according to claim 1, wherein the second connection part has a length in the circumferential direction, which is a length smaller than a length of the first connection part in the circumferential direction.

4. The ultrasound endoscope according to claim 1, wherein the second connection part has a length in the circumferential direction, which is a length larger than a length of the first connection part in the circumferential direction.

5. The ultrasound endoscope according to claim 3, further comprising an electrode that is formed in the second connection part and that is connected to the ultrasound cable,
wherein a longitudinal direction of the electrode is parallel with a width direction of the t is formed in the second connection part, the electrode is formed along a direction orthogonal to the width direction, and the second connection part has a width larger than that of the joint.

6. The ultrasound endoscope according to claim 1, wherein the flexible substrate includes a first flexible substrate and a second flexible substrate each of which includes the first connection part, the second connection part, and the joint, and
joints of the first flexible substrate and the second flexible substrate overlap partly.

7. The ultrasound endoscope according to claim 6, wherein the second connection part of the first flexible substrate and the second connection part of the second flexible substrate are aligned along the longitudinal direction.

8. The ultrasound endoscope according to claim 1, wherein the second connection part forms a zigzag along a longitudinal direction.

9. The ultrasound endoscope according to claim 1, further comprising a plurality of electrodes that are connected to the coaxial cores, respectively, in the second connection part,
wherein the electrodes are formed on a surface of the flexible substrate on one side.

10. The ultrasound endoscope according to claim 1, further comprising a plurality of electrodes that are connected to the coaxial cores, respectively, in the second connection part,
wherein the electrodes are formed on surfaces of the flexible substrate on both sides.

11. The ultrasound endoscope according to claim 1, wherein the flexible substrate includes a plurality of first electrodes that are electrically connected to the piezoelectric elements, respectively, and a plurality of second electrodes that are electrically connected to the first electrodes and the coaxial cores, respectively, and
a longitudinal direction of each of the second electrodes on a surface of connection is oblique along a direction in which cores of the coaxial cores enter.

12. The ultrasound endoscope according to claim 1, wherein in a cross section passing through an end of the jacket in the distal-end rigid part, a straight line passing through a center of the ultrasound cable and a center of the channel passes through a center axis of the insertion part.

13. The ultrasound endoscope according to claim 12, wherein the straight line is parallel with a direction corresponding to a vertical direction of an image that is captured by the imaging sensor.
